(19) United States
(12) Patent Application Publication
(10) Pub. No.: US 2012/0135507 A1
(43) Pub. Date: May 31, 2012

(54) BIOCHIP MODULE WITH CERAMIC LAMINATE STRUCTURE AND METHOD OF MANUFACTURING THE SAME

(75) Inventors: Jong-Hee KIM, Seoul (KR); Jeong-Ho CHANG, Gwangmyeong-si (KR); Dong-Hun YEO, Seoul (KR); Young-Joon YOON, Yongin-si (KR); Sang-Il HYUN, Seoul (KR); Bong-Yong JUNG, Yeonsu-gu (KR); Jin-Hyung LEE, Hwaseong-si (KR)

(73) Assignee: KOREA INSTITUTE OF CERAMIC ENGINEERING AND TECHNOLOGY, Seoul (KR)

(21) Appl. No.: 13/301,174

(22) Filed: Nov. 21, 2011

(30) Foreign Application Priority Data
Nov. 30, 2010 (KR) 10-2010-0120954

(51) Int. Cl. C12M 1/34; B23P 11/00

(52) U.S. Cl. 435/287.1; 137/15.01

(57) ABSTRACT

The present disclosure provides a biochip module having a ceramic laminate structure which uses advantages of ceramic and enables a reduction in a chip area, and a method of manufacturing the same. The biochip module includes a first ceramic layer mixing bacterial water with magnetic beads to which ligands capturing bacteria are attached, a second ceramic layer separating the magnetic beads capturing bacteria from the water, and a third ceramic layer detecting the number of bacteria captured by the magnetic beads.

### CROSS-REFERENCE TO RELATED APPLICATION

This application claims the benefit under 35 U.S.A. §119 of Korean Patent Application No. 10-2010-0120954, filed on Nov. 30, 2010 in the Korean Intellectual Property Office, the entirety of which is incorporated herein by reference.

### BACKGROUND

1. Technical Field

The present invention relates to a method of manufacturing a biochip module capable of detecting bacteria for environmental monitoring, and more particularly, to a biochip module with a ceramic laminate structure which includes ceramic as a chip material and enables a reduction of a chip area, and a method of manufacturing the same.

2. Description of the Related Art

Currently, ceramics have a wide range of applications in industry including electric and electronic fields, aerospace, bio-industry, and the like.

Such ceramics have various merits including biomaterial capturing performance and chemical resistance, and thus may be applied to manufacture of a biochip used to detect bacteria, such as colon bacilli.

Table 1 shows characteristics of ceramics as a biochip material.

TABLE1

| Kind | | Metal | Polymer | Ceramic |
|---|---|---|---|---|
| Biomaterial | Direct capturing | X | Δ | ◯ |
| capturing | (Physorption) | | | |
| performance | Chemisorption | ◯ | ◯ | ◯ |

| | | | |
|---|---|---|---|
| Heat resistance | ◯ | Δ | ◯ |
| Chemical resistance | ◯ | Δ | ◯ |
| Mass productivity (unit cost) | X | ◯ | ◯ |
| Increase in surface area | X | Δ | ◯ |
| (Fine structure control) | | | |
| Error in signal conversion by | Medium | High | Low |
| external environment | | | |
| Electrical conductivity | ◯ | Δ | Δ |
| Reusability | Δ | Δ | ◯ |

As shown in Table 1, ceramics have superior properties to metals or polymers in almost every aspect in terms of biomaterial capturing performance, heat resistance, chemical resistance, fine structure controlling properties, signal conversion error by an external environment, reusability, and the like.

Therefore, there is a need for technology for manufacturing a biochip using ceramics having such advantages.

### BRIEF SUMMARY

The present invention provide a biochip module with a ceramic laminate structure which includes ceramic as a chip material and enables a reduction in a chip area, thereby enabling application to various fields, such as bacteria (microorganism) detection.

The present invention also provides a method of manufacturing a biochip through stacking ceramic layers.

An aspect of the present invention relates to a biochip module, which includes: a first ceramic layer mixing bacterial water with magnetic beads to which ligands capturing bacteria are attached; a second ceramic layer separating the magnetic beads capturing bacteria from the water; and a third ceramic layer detecting the number of bacteria captured by the magnetic beads.

Here, the number of bacteria captured by the magnetic beads may be detected in an electric mode or in a fluorescent mode.

Another aspect of the invention relates to a method of manufacturing a biochip module, which includes: stacking a first ceramic layer including a first channel to mix bacterial water with magnetic beads to which ligands capturing bacteria are attached, a second ceramic layer including a second channel to separate the magnetic beads capturing bacteria from the water, and a third ceramic layer including a third channel to transfer the magnetic beads capturing the bacteria and a detector to detect the number of bacteria captured by the magnetic beads, such that an inlet of the second channel is connected to an outlet of the first channel and an inlet of the third channel is connected to an outlet of the second channel.

Here, the method of manufacturing the biochip module further includes forming ceramic sheets respectively corresponding to the first ceramic layer, the second ceramic layer, and the third ceramic layer using tape casting, forming channels in the ceramic sheets, and stacking and sintering the ceramic sheets together.

Further, the channel formed in each of the ceramic sheets may be formed using a photoresist.

According to embodiments of the present invention, a biochip module may be formed to have a ceramic laminate structure through a series of processes of mixing bacterial water and magnetic beads, separating the magnetic beads capturing bacteria, and detecting the number of bacteria captured by the magnetic beads.

The laminate structure of the biochip module enables a reduction in a chip size, so that the biochip module may be manufactured to a small size.

In addition, the biochip module having the ceramic laminate structure may be easily manufactured through LTCC technology, which includes forming ceramic sheets using tape casting, forming channels in the ceramic sheets using a photoresist, and stacking and sintering the ceramic sheets together.

### BRIEF DESCRIPTION OF THE DRAWINGS

The above and other aspects, features, and advantages of the invention will become apparent from the following detailed description of exemplary embodiments in conjunction with the accompanying drawings, in which:

FIG. 1 is a schematic view of a biochip module for detecting bacteria;

FIG. 2 is a schematic view of a biochip module according to an exemplary embodiment of the present invention;

FIGS. 3 and 4 are schematic views of examples of channels in a zigzag shape formed on a first ceramic layer; and

FIG. 5 is a flowchart of a method of manufacturing a biochip module using low temperature co-fired ceramic (LTCC) technology according to an exemplary embodiment of the present invention.

### DETAILED DESCRIPTION

Exemplary embodiments of the invention will be described in detail with reference to the accompanying drawings. It should be understood that the present invention is not limited to the following embodiments and may be embodied in different ways, and that the embodiments are given to provide complete disclosure of the invention and to provide thorough understanding of the invention to those skilled in the art. The scope of the invention is limited only by the accompanying claims and equivalents thereof. Like components will be denoted by like reference numerals throughout the specification and the accompanying drawings.

Hereinafter, a biochip module with a ceramic laminate structure and a method of manufacturing the same according to exemplary embodiments of the present invention will now be described in detail with reference to the accompanying drawings.

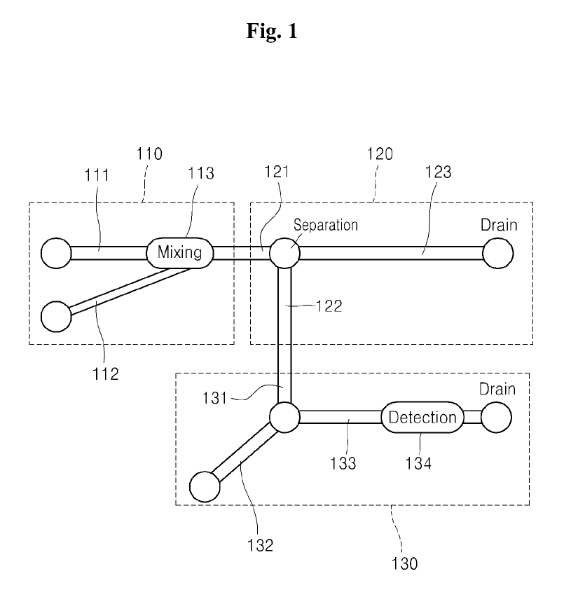

FIG. 1 schematically illustrates a biochip module for detecting bacteria.

Referring to FIG. 1, the biochip module includes a mixing unit 110, a separation unit 120, and a detection unit 130.

The mixing unit 110 mixes bacterial water with magnetic beads, to which ligands capturing bacteria are attached, such that bacteria included in the bacterial water are captured by the ligands of the magnetic beads.

The mixing unit 110 includes a 1-1 channel 111 through which the bacterial water is supplied, a 1-2 channel 112 through which the magnetic beads are supplied, a 1-3 channel 113 on which the 1-1 channel 111 and the 1-2 channel 112 converge.

The separation unit 120 separates magnetic beads capturing bacteria from water.

The separation unit 120 includes a 2-1 channel 121 transferring a mixture of water and magnetic beads capturing bacteria, a 2-2 channel 122 transferring the magnetic beads capturing bacteria, and a 2-3 channel 123 through which the water separated from the magnetic beads is drained.

The detection unit 130 detects bacteria captured by the magnetic beads, particularly the number of bacteria captured by the ligands attached to the magnetic beads.

The detection unit 130 includes a 3-1 channel 131 transferring the magnetic beads capturing the bacteria, a 3-2 channel 132 providing a detection buffer including a detection reagent reacting with bacteria to provide a fluorescent signal, a 3-3 channel 133 in which a detector 134 is disposed to detect bacteria captured by the magnetic beads.

As such, the biochip module may conduct all processes of mixing bacterial water with magnetic beads to which ligands capturing bacteria are attached, separating the magnetic beads capturing bacteria, and detecting bacteria captured by the magnetic beads.

However, as shown in FIG. 1, when a biochip module is manufactured on a two-dimensional plane, the biochip module has a large size.

Such a problem can be solved by forming a biochip module having a ceramic laminate structure.

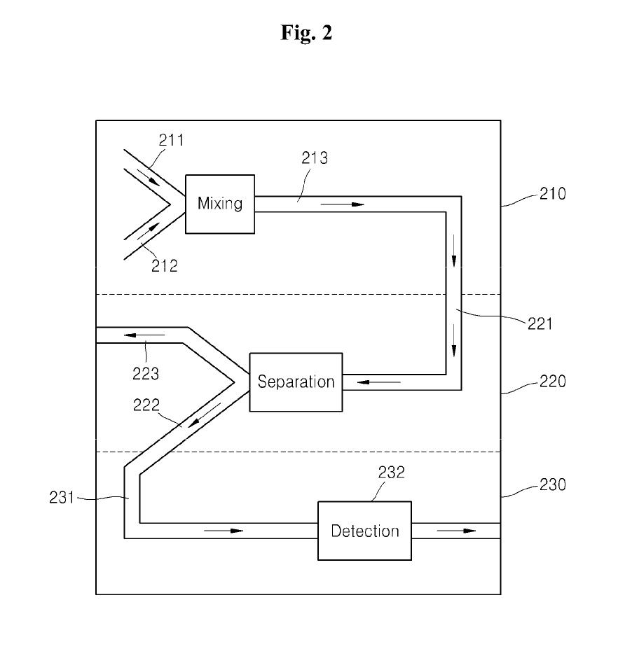

FIG. 2 is a schematic view of a biochip module according to an exemplary embodiment of the present invention.

Referring to FIG. 2, the biochip module includes a first ceramic layer 210, a second ceramic layer 220, and a third ceramic layer 230.

First Ceramic Layer

The first ceramic layer 210 serves to mix bacterial water with magnetic beads to which ligands capturing bacteria are attached.

The first ceramic layer 210 includes a first channel providing a space in which the bacterial water and the magnetic beads are mixed such that the bacteria included in the bacterial water are captured by the magnetic beads.

In detail, the first ceramic layer 210 may include a 1-1 channel 211, a 1-2 channel 212, and a 1-3 channel 213. These channels 211, 212, 213 may be formed on the same plane.

Through the 1-1 channel 211, bacterial water is supplied. The bacterial water includes bacteria, such as colon bacilli.

Through the 1-2 channel 212, the magnetic beads which the ligands capturing bacteria are attached to are supplied. Here, the magnetic beads may have a nano scale diameter of about 10 to 500 nm. For example, the magnetic beads may be magnetic silica beads, and the ligands capturing bacteria may be attached to the magnetic silica beads, for example, by cross linkage through N-(3-dimethylaminopropyl)-N-ethylcarbodiimide hydrochloride (EDC) and N-hydroxysuccinimide (NHS).

The 1-3 channel 213 is formed by joining the 1-1 channel 211 and the 1-2 channel 212. That is, in the 1-3 channel 213, the bacterial water supplied through the 1-1 channel 211 and the magnetic beads supplied through the 1-2 channel 212 are mixed, so that the bacteria included in the bacterial water are captured by the magnetic beads.

Here, the 1-3 channel 213 may be formed in a zigzag shape rather than in a straight line so that the channel is long. In this case, a degree of mixing the bacterial water with the magnetic beads may increase due to a long length of the 1-3 channel 213. Thus, the bacteria included in the bacterial water may be captured as much as possible by the ligands attached to the magnetic beads.

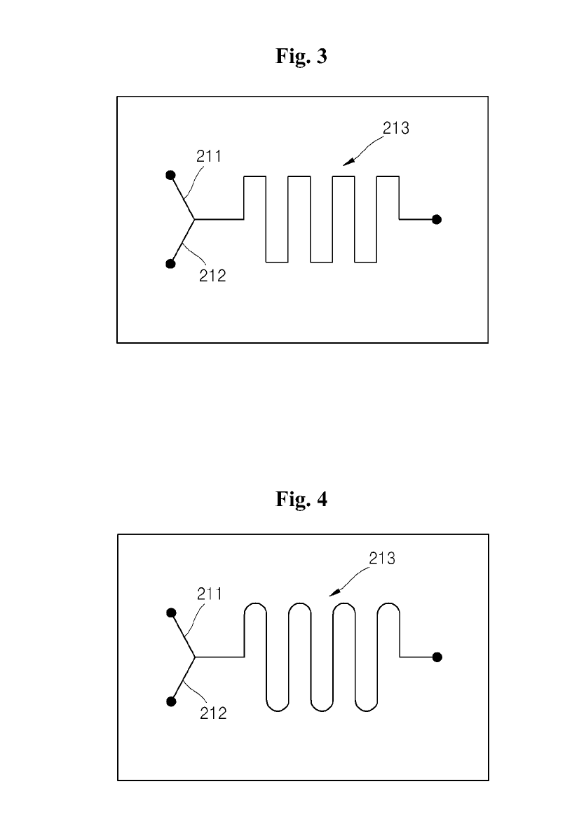

The 1-3 channel 213 of the zigzag shape may have an angularly bent section, as shown in FIG. 3. Alternatively, the 1-3 channel 213 of the zigzag shape 213 may have a curvedly bent part, as shown in FIG. 4. In addition to these examples shown in FIGS. 3 and 4, the 1-3 channel 213 of the zigzag shape may have various modifications.

Second Ceramic Layer

The second ceramic layer 220 serves to separate the magnetic beads having the ligands capturing bacteria from the water.

The second ceramic layer 220 includes a second channel connected to the first channel formed in the first ceramic layer 210 and providing a space in which the magnetic beads capturing bacteria are separated from the water.

In detail, the second ceramic layer 220 may include a 2-1 channel 221, a 2-2 channel 222, and a 2-3 channel 223. These channels 221, 222, and 223 may be formed on the same plane.

The 2-1 channel 221 has an inlet connected to an outlet of the 1-3 channel 213 of the first ceramic layer 210. Accordingly, a mixture of the magnetic beads capturing bacteria and the water is transferred through the 2-1 channel 221.

The 2-2 channel 222 diverges from the 2-1 channel 221. Through the 2-2 channel 222, the magnetic beads capturing bacteria are transferred. Here, through the 2-2 channel 222, only bacteria captured by the magnetic beads may be transferred or the magnetic beads capturing bacteria may be transferred as a concentrate.

The 2-3 channel 223 diverges from the 2-1 channel 221 in a different direction from that of the 2-2 channel 222. Through the 2-3 channel 223, water separated from the magnetic beads capturing the bacteria and water which does not contain bacteria or has a remarkably reduced concentration of bacteria is drained.

The second ceramic layer 220 may include a magnet, such as an electromagnet and a permanent magnet, in order to induce the magnetic beads to move toward the 2-2 channel.

Third Ceramic Layer

The third ceramic layer 230 serves to detect the number of bacteria captured by the magnetic beads.

The third ceramic layer 230 includes a third channel 231 which is connected to the second channel in the second ceramic layer 220 and in which the magnetic beads capturing bacteria are transferred and the number of bacteria captured by the magnetic beads is detected.

The third channel 231 is formed with a detector 232 to detect the number of bacteria captured by the magnetic beads.

The detector 232 may detect the number of bacteria captured by the magnetic beads in an electric mode of sensing a change in resistance or electric current according to the number of bacteria or in a fluorescent mode of measuring a fluorescent signal according to the number of bacteria.

In the fluorescent mode, the detector 232 may use a detection buffer including a detection reagent which reacts with bacteria to provide a fluorescent signal.

The detection buffer may be supplied to the third channel 231 through a detection buffer supply channel 132, as shown in FIG. 1.

In the electric mode, the detector 232 includes an electrode (not shown), on which other ligands capturing bacteria captured by the magnetic beads are fixed, thereby detecting an electric signal generated by the bacteria captured by the magnetic beads.

A value detected by the detector 232 may be used by the biochip module or may be transmitted to a regional central control center through wireless communication.

Referring to FIG. 2, the biochip module is formed by sequentially stacking the first ceramic layer 210, the second ceramic layer 220, and the third ceramic layer 230.

Although not shown, the biochip module may be formed in order of the first ceramic layer 210, the third ceramic layer 230, and the second ceramic layer 220. In this case, the first channel of the first ceramic layer 210 and the second channel of the second ceramic layer 220 may be connected via a through-channel (not shown) penetrating the third ceramic layer 230, instead of being directly connected to each other.

The biochip module according the embodiment of the invention includes ceramic and thus maintains natural advantages of ceramic in terms of biomaterial capturing performance, heat resistance, chemical resistance, fine structure controlling properties, signal conversion error by an external environment, and reusability, as listed in Table 1.

Further, the biochip module embodiment of the invention has a ceramic laminate structure enabling a reduction in size thereof.

The biochip module having a laminate structure of the first, second and third ceramic layers may be easily manufactured by stacking the layers such that an inlet of the second channel (specifically, the 2-1 channel) formed in the second ceramic layer is connected to an outlet of the first channel (specifically, the 1-3 channel) and an inlet of the third channel formed in the third ceramic layer is connected to an outlet of the second channel (specifically, the 2-2 channel) of the second ceramic layer.

Stacking may be carried out in order of the first ceramic layer, the second ceramic layer, and the third ceramic layer.

Alternatively, stacking may be carried out in order of the first ceramic layer, the third ceramic layer, and the second ceramic layer. In this case, a through-channel may be formed in the third ceramic layer to connect the first channel to the second channel.

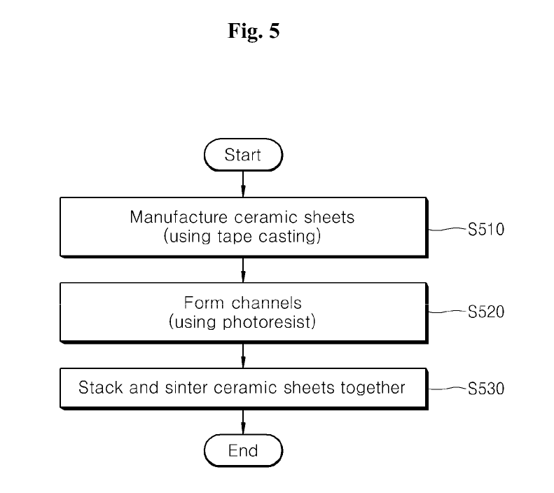

The biochip module having the laminate structure of the first, second and third ceramic layers may be manufactured by low temperature co-fired ceramic (LTCC) technology, as shown in FIG. 5.

FIG. 5 is a flowchart of a method of manufacturing a biochip module using LTCC technology according to an exemplary embodiment of the invention. A biochip module having a ceramic laminate structure may be manufactured by LTCC technology as follows.

First, ceramic sheets respectively corresponding to a first ceramic layer, a second ceramic layer, and a third ceramic layer are formed using tape casting (S510).

Then, channels are formed in the ceramic sheets (S520). Each channel may have a width of about 200 to 500 μm and a depth of about 150 to 250 μm.

The channels may be formed using a photoresist through exposure, development, and the like.

Since formation of the channels is conducted before sintering in a state that the ceramic sheets are not completely cured, the channel may be easily formed through a photoresist process.

Then, the ceramic sheets are aligned, stacked, and sintered together at an LTCC sintering temperature of about 800 to 1,000° C. (S530).

Therefore, the biochip module having the laminate structure may be easily manufactured through LTCC technology.

Although some embodiments have been described herein, it should be understood by those skilled in the art that these embodiments are given by way of illustration only, and that various modifications, variations, and alterations can be made without departing from the spirit and scope of the invention. Therefore, the scope of the invention should be limited only by the accompanying claims and equivalents thereof.

What is claimed is:

1. A biochip module comprising:
a first ceramic layer mixing bacterial water with magnetic beads to which ligands capturing bacteria are attached;
a second ceramic layer separating the magnetic beads capturing bacteria from the water; and
a third ceramic layer detecting the number of bacteria captured by the magnetic beads.

2. The biochip module of claim 1, wherein the first ceramic layer comprises a first channel in which the bacterial water and the magnetic beads are mixed such that the bacteria included in the bacterial water are captured by the magnetic beads, the second ceramic layer comprises a second channel which is connected to the first channel and in which the magnetic beads capturing bacteria are separated from the water, and the third ceramic layer comprises a third channel which is connected to the second channel and in which the magnetic beads capturing the bacteria are transferred and the number of bacteria captured by the magnetic beads is detected.

3. The biochip module of claim 2, wherein the biochip module is constituted by sequentially stacking the first ceramic layer, the second ceramic layer, and the third ceramic layer.

4. The biochip module of claim 2, wherein the biochip module is constituted by sequentially stacking the first ceramic layer, the third ceramic layer, and the second ceramic layer, the first channel and the second channel being connected to each other via a through-channel penetrating the third ceramic layer.

5. The biochip module of claim 2, wherein the first ceramic layer comprises a 1-1 channel through which the bacterial water is supplied, a 1-2 channel through which the magnetic beads are supplied, and a 1-3 channel in which the bacterial water supplied from the 1-1 channel and the magnetic beads supplied from the 1-2 channel are mixed such that bacteria included in the bacterial water are captured by the magnetic beads.

6. The biochip module of claim 5, wherein the 1-3 channel is formed in a zigzag shape.

7. The biochip module of claim 2, wherein the second channel comprises a 2-1 channel through which a mixture of the magnetic beads capturing the bacteria and the water is transferred, a 2-2 channel which diverges from the 2-1 channel and through which the magnetic beads capturing the bacteria are transferred, and a 2-3 channel which diverges from the 2-1 channel and through which the water separated from the magnetic beads capturing the bacteria is drained.

8. The biochip module of claim 7, wherein the second ceramic layer comprises a magnet inducing the magnetic beads to move toward the 2-2 channel.

9. The biochip module of claim 2, wherein the third channel comprises a detector detecting the number of bacteria captured by the magnetic beads.

10. The biochip module of claim 9, wherein the detector detects the number of bacteria captured by the magnetic beads in an electric mode or in a fluorescent mode.

11. The biochip module of claim 10, wherein the detector detects the number of bacteria using a detection buffer including a detection reagent which reacts with the bacteria captured by the magnetic beads to provide a fluorescent signal.

12. The biochip module of claim 11, wherein the detection buffer is supplied through a detection buffer supply channel formed in the third ceramic layer and connected to the third channel.

13. The biochip module of claim 10, wherein the detector comprises an electrode, to which other ligands to combine with the bacteria captured by the magnetic beads are attached, such that the detector detects the number of bacteria using a change in an electric signal generated by bacteria combining with the ligands attached to the electrode.

14. A method of manufacturing a biochip module, comprising:
stacking a first ceramic layer including a first channel to mix bacterial water with magnetic beads to which ligands capturing bacteria are attached, a second ceramic layer including a second channel to separate the magnetic beads capturing bacteria from the water, and a third ceramic layer including a third channel to transfer the magnetic beads capturing the bacteria and a detector to detect the number of bacteria captured by the magnetic beads, such that an inlet of the second channel is connected to an outlet of the first channel and an inlet of the third channel is connected to an outlet of the second channel.

15. The method of claim 14, wherein the second ceramic layer is stacked on the first ceramic layer, and the third ceramic layer is stacked on the second ceramic layer.

16. The method of claim 14, wherein the third ceramic layer is stacked on the first ceramic layer, and the second ceramic layer is stacked on the third ceramic layer, the third ceramic layer being formed with a through-channel through which the first channel is connected to the second channel.

17. The method of claim 14, wherein the first ceramic layer comprises a 1-1 channel through which the bacterial water is supplied, a 1-2 channel through which the magnetic beads are supplied, and a 1-3 channel in which the bacterial water supplied from the 1-1 channel and the magnetic beads supplied from the 1-2 channel are mixed such that bacteria included in the bacterial water are captured by the magnetic beads.

18. The method of claim 14, wherein the second ceramic layer comprises a 2-1 channel through which a mixture of the magnetic beads capturing the bacteria and the water is transferred, a 2-2 channel which diverges from the 2-1 channel and through which the magnetic beads capturing the bacteria are transferred, and a 2-3 channel which diverges from the 2-1 channel and through which the water separated from the magnetic beads capturing the bacteria is drained.

19. The biochip module of claim 14, wherein the third ceramic layer comprises a detection buffer channel which is connected to the third channel and through which a detection buffer is supplied and attached to the bacteria captured by the magnetic beads.

20. The method of claim 14, further comprising:
forming ceramic sheets respectively corresponding to the first ceramic layer, the second ceramic layer, and the third ceramic layer using tape casting;
forming channels in the ceramic sheets using a photoresist; and
stacking and sintering the ceramic sheets together.
